(19) United States
(12) Patent Application Publication
(10) Pub. No.: US 2022/0347450 A1
(43) Pub. Date: Nov. 3, 2022

(54) MICRONEEDLE, MICROCONE, AND PHOTOLITHOGRAPHY FABRICATION METHODS

(71) Applicants: KANSAS STATE UNIVERSITY RESEARCH FOUNDATION, Manhattan, KS (US); RAPHAS CO., LTD, Seoul (KR)

(72) Inventors: Jungkwun KIM, Manhattan, KS (US); Keun Ho LEE, Seoul (KR); Jung Dong KIM, Seoul (KR); Dohyeon JEONG, Seoul (KR)

(21) Appl. No.: 17/862,315

(22) Filed: Jul. 11, 2022

Related U.S. Application Data
(63) Continuation of application No. PCT/US2021/013629, filed on Jan. 15, 2021.
(60) Provisional application No. 62/961,931, filed on Jan. 16, 2020.

(51) Int. Cl. A61M 37/00; G03F 7/00; G03F 7/20

(52) U.S. Cl. CPC A61M 37/0015; G03F 7/0037; G03F 7/203; B33Y 10/00

(57) ABSTRACT

Lithography fabrication methods for producing polymeric microneedles, microprobes, and other micron-sized structures with sharp tips. The fabrication process utilizes a single-step bottom-up exposure of photosensitive resin through a photomask micro-pattern, with a corresponding change/increase in refractive index of the resin creating a meta-state waveguide within the resin which focuses down additional transmitted energy and forms a converging shape (first harmonic microcone). Energy is diffracted through the tip of the first harmonic microcone as a second harmonic beam to form a second converging shape (second harmonic shape) adjacent the first microcone, followed by additional tertiary harmonic microcones, which can be built upon these structures with application of additional energy.

### CROSS-REFERENCE TO RELATED APPLICATIONS

The present application is a Continuation of PCT/US2021/013629 filed on Jan. 15, 2021, which claims priority to Provisional Application No. 62/961,931 filed on Jan. 16, 2020. The aforementioned applications are incorporated herein by reference in their entireties.

### BACKGROUND

### Technical Field

The present invention relates to new photolithography techniques for fabricating microstructures, particularly microcones and/or microneedles.

### 2. Description of Related Art

The development of microneedles has been long established since 1990, and many studies have shown that microneedles have great advantages in drug delivery over oral administration and hypodermic needle injection. Microneedles typically have a sharper tip than hypodermic needles and a height of only 10-2000 μm, providing a minimally invasive way of drug delivery. Oral administration is convenient but suffers from low drug delivery efficiency due to drug degradation and poor absorption in the human body. The transdermal drug delivery method also faces issues that many drugs cannot pass through the outermost skin layer, resulting in low delivery efficiency. Recent reports, however, have shown that microneedles are able to penetrate the skin and deliver drugs into the epidermis and or dermis layer without pain.

The geometry of microneedles plays an important role in the insertion behavior and mechanical stability of microneedles. A sharp microneedle tip with a small taper angle and diameter reduces the insertion force but increases the possibility of fracture and buckling failure. A recent study has reported some common types of microneedle based on taper angle at the tip and the needle body, and their corresponding average insertion forces into a chicken breast. The study concluded that a needle tip with isosceles triangle geometry with a taper angle of 30° is the optimal needle tip shape as it showed the highest durability against buckling force among four types of geometries as well as moderate average insertion force without fracture failure. However, the 3-D formation of non-straight geometry such as curved or tapered shape requires a layer-by-layer forming process or multiple photomask alignment processes, which could potentially increase the fabrication time as well as manufacturing cost.

### SUMMARY

The present disclosure leverages optical diffraction associated with liquid-to-solid crosslinking, which forms a light waveguide due to different refractive indices of uncrosslinked and crosslinked resin, enabling the formation of various types of micro-cone structures in a rapid and straightforward process within 30 minutes including UV exposure and developing processes.

The proposed microneedle fabrication method is exclusively advantageous as different photomask patterns generate various microneedle shapes including circular, star, hexagonal, and triangle bases as well as advanced functional microneedles such as hollow and inclined microneedles. As the conventional UV lithography approach to fabricate the mentioned microneedles requires multiple UV exposure and alignment processes, a versatile and straightforward fabrication process should be carefully addressed to be a low-cost sophisticated drug delivery product.

The present invention is broadly concerned with novel fabrication methods for producing microneedles and other micron-sized structures with sharp tips. The fabrication process utilizes bottom-up exposure of a liquid photosensitive resin through a photomask pattern comprising a plurality of apertures, for example 200 μm-diameters holes, or other shapes. A UV light is exposed through the photomask pattern. Exposed photosensitive resin polymerizes and grows into micro structures with sharp tips. The refractive index of the material becomes higher compared to the surrounding liquid state photosensitive resin. The later formed refractive index contrast between the solid and the liquid resin causes UV light reflection at the boundary like a light waveguide and sends the light to the vertex of the cone. That is, once the liquid resin becomes solid, the solidified region works as a light waveguide to focus down additional transmitted light and form a cone shape (first harmonic microcone). Further UV exposure causes the UV light to radiate through the vertex of the cone to form a small sharp tip. The light diffracted again through the tip as a second harmonic beam with even further exposure to form a second cone shape (second harmonic shape). The third cone is built along the same principle with a smaller size due to the lower light intensity. We observed up to the 4th cone in the experiments.

Unlike prior work using solid resins, which aimed at forming vertical sidewalls, the current work using liquid resins yields converging or tapered sidewalls, which form micro-needle type structures or structures with inclined sidewall angles. The fabricated structures are rinsed with solvent to remove unreacted composition. The fabricated structures can be used as microneedles and microprobes.

In one aspect, there is provided a method for fabricating a plurality of micro-sized structures with converging tips, the method comprising: providing a substrate having an upper surface and a backside surface, wherein said substrate comprises a pattern having open areas configured to permit radiation to pass through the substrate and solid areas configured to prevent radiation from passing through the substrate; forming a layer of liquid-state photosensitive resin on said upper surface; exposing said liquid-state photosensitive resin to radiation through said substrate from the backside surface over a first period of time to yield light-exposed portions of said liquid-state photosensitive resin, wherein said light-exposed portions are crosslinked and/or polymerized into respective solid-state resin structures on said upper surface in alignment with said open areas, said solid-state resin structures having an increased refractive index as compared to said liquid-state photosensitive resin, such that each solid-state resin structure acts as a waveguide directing said radiation passing through said open areas of said substrate to a converging point thereby forming solid-state resin structures with tapered sidewalls and converging tips; and contacting the coating layer with a solvent system so as to remove non-light exposed portions of said liquid-state photosensitive resin to leave behind a plurality of micro-sized solid-state resin structures with converging tips across said upper surface of said substrate.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustration (not to scale) of an exemplified process for forming micro-structures;

FIG. 2 is a further illustration showing conical light profile of the UV light propagation through micro-sized apertures to polymerize the liquid photosensitive resin and development of additional harmonic structures over time as more energy propagates through the resin;

FIG. 3 is an image from the UV diffraction experimental validation visualizing light propagation inside the liquid photosensitive resin using a photomask with a pattern size of 200 μm;

FIG. 4 shows several photographs of microcone fabrication using conditions from Table 1;

FIG. 5 shows SEM images of the second harmonic microcone using a 120 p.m photopattern base, 884 μm tall, 50 μm “waist” (left panel), and a close-up view of the second harmonic cone (right panel);

FIG. 6A illustrates light intensity distribution of collimated UV light with a Gaussian profile after passing through a photomask with circular aperture;

FIG. 6B illustrates light intensity distribution with a Gaussian profile after passing through a photomask with circular aperture with opaque core for hollow shape formation;

FIG. 7 is a graph of the measured UV intensity (375 nm) at 0.5 inches above the light source attenuated by no glass (dots), one glass (triangles) and two glasses (squares);

FIG. 8 shows SEM images of (a) one unit of microneedle and (b) enlarged view at the tip;

FIG. 9 shows images of micro-structure with multiple harmonics: (a) fabricated microneedle, (b) UV light propagation, micro-structure array with (c) secondary harmonic and (d) tertiary harmonic;

FIG. 10 is an image showing an array of micro-structures with various base geometries and heights, fabricated with a single simultaneous exposure, and the corresponding patterned photomask (inset) that was used;

FIG. 11 is a graph of the relationship between the height of fabricated microneedles and the applied energy, secondary y-axis refers to the aspect ratio of the corresponding height and secondary x-axis refers to the exposure time of the corresponding applied energy;

FIG. 12 shows photographic images of micro-structures at various applied energy which corresponds to (a) 2 seconds, (b) 3 seconds, (c) 5 seconds, and (d) 20 seconds of exposure time and the resulting exposure dosage;

FIG. 13 shows photographic images of (a) PLA microneedle array fabricated using PDMS micromolding from diffraction lithography micro-structure template; and (b) pigskin with insertion marks;

FIG. 14 shows the results of the micro-structures force-displacement test: tip of the needle deformed while the body remained durable;

FIG. 15 shows images of a 3×3 circular micro-structures array: (a) conical light profile of the UV light propagation (b) the corresponding microneedles (SEM);

FIG. 16 is a graph showing the relationship of micro-structures height at different applied energy; (secondary x-axis) exposure time; (secondary y-axis) aspect ratio

FIG. 17 shows the SEM pictures of microneedle array with various base geometries (inset): (a) circular, (b) hexagonal, (c) triangular, and (d) star;

FIG. 18 shows the SEM pictures of the (a) hollow microneedle array and (b) inclined circular microneedle array;

FIG. 19 shows photographs from the insertion test result: 3x3 PLA circular microneedle array of insertion marks on pigskin, including inset images of the microneedles before and after insertion;

FIG. 20A are SEM images from the force-displacement test result of a 3×3 PLA circular microneedle array: before insertion (top panel), tip broken (middle panel), and body broken (bottom panel);

FIG. 20B is a graph of the data from the force-displacement test;

FIG. 21 is a graph of the minimum crosslinking energy for surgical guide resin at various light intensity with 405nm UVLED;

FIG. 22 is a graph of the measured transmission of 405 nm UV light through various thickness of surgical guide resin;

FIG. 23 is a graph of the measured height of the crosslinked resin at various energy;

FIG. 24 is an illustration of the experimental set up for solid microneedle fabrication in Example 4;

FIG. 25 is a graph of the measured height of the microneedles over various exposure energy and time;

FIG. 26 shows photographs of micro-cone and microneedles with first, second and third harmonics corresponding to the labeled exposure energy in the previous figure;

FIG. 27A is an SEM image of a 20×20 array of solid perpendicular microneedles;

FIG. 27B is an enlarged SEM image of an individual microneedle from FIG. 27A;

FIG. 27C is a further enlarged SEM image of the tip of the individual microneedle from FIG. 27B;

FIG. 28 is an illustration of the experimental set up for hollow microneedle fabrication in Example 4;

FIG. 29 is a photograph of an array of 271 units of hollowed-through microneedles with base diameter of 280 μm and height of 550 μm—inset is the image of the annular photomask pattern used;

FIG. 30 is a photograph of a close-up image of three different hollowed-through microneedles from FIG. 29 showing the high consistency of the shape profile across the array;

FIG. 31 shows the result of the insertion test and an inset SEM image of the microneedle array used;

FIG. 32A shows the data from the force-displacement test in Example 4 of the perpendicular microneedles;

FIG. 32B shows the images of the perpendicular microneedles from FIG. 32A, showing (a) before; (b) needle tip broken; and (c) body broken;

FIG. 33A shows the data from the force-displacement test in Example 4 of the angled microneedles;

FIG. 33B shows the images of the angled microneedles from FIG. 33A, showing (a) before; (b) needle tip broken; and (c) detachment from the substrate;

FIG. 34A shows the data from the in-phase and out-phase force application on the angled microneedles from Example 4; and

FIG. 34B shows images depicting the direction of application of force for the out-phase and in-phase tests.

### DETAILED DESCRIPTION

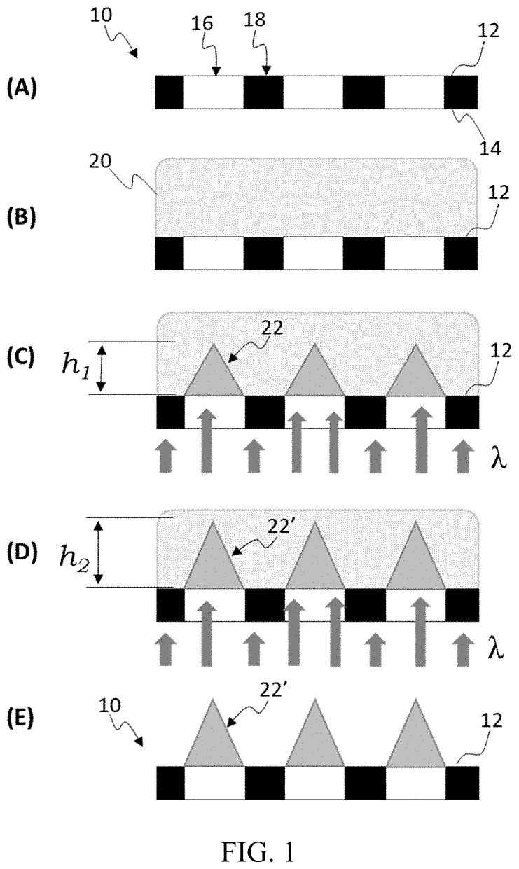

In more detail, with reference to FIG. 1, the process involves providing a generally planar substrate 10 having an upper surface 12and a backside surface 14. The substrate 10 is typically transparent or essentially transparent to permit transmission of activating radiation through the substrate 10. Appropriate substrates include glass, fused silica, polymers or plastics (acrylics, plexiglass, etc.), and the like. The substrate 10 further includes a pattern (e.g., photomask) having open areas 16 configured to permit radiation to pass and solid (opaque) areas 18 configured to prevent or block radiation from passing through. The pattern may be integrally formed as part of the substrate itself, as shown in FIG. 1, or the pattern may be a separate patterned layer adjacent the upper surface and/or backside surface of the substrate. In one or more embodiments, the pattern may comprise an array of spaced-apart apertures (windows) distributed across the surface. It will be appreciated that the geometric shape and size (e.g., width or diameter) of the apertures can be designed as desired to generate microstructures having the desired geometric shape, as discussed in more detail below. Typically, in the context of the present disclosure, the apertures are micro-sized meaning that they have a maximum size of up to 1,000 μm, where “size” refers to the maximum edge-to-edge dimension (e.g., diameter in the case of circular apertures, maximum width in the case of rectangular apertures, or point-to-point in the case of stars).

As depicted in FIG. 1(B), a liquid-state photosensitive resin 20 is then applied to the upper surface 1 2of the substrate 10 to create a coating layer thereon. The photosensitive resin 20 is preferably applied at a thickness (as measured from the upper surface 12 of the substrate 10) that is greater than the desired height of the structures to be formed. In general, the thickness of the coating layer will range from about 50 μm to about 9 mm. As used herein “resin” refers to various monomeric, oligomeric, and/or polymeric compositions, which typically comprise monomers, oligomers, and/or polymers dispersed in a solvent system along with optional photoinitiators. Such photosensitive resins are known in the art, including compositions conventionally used for negative-tone photoresists in microelectronic fabrication, as well as resins for 3D printing. Exemplary resins include various epoxies, acrylates, polyurethanes, methacrylated oligomers, monomers, or polymers, urethane methacrylates, diphenyl (2,4,6-trimethylbenzoyl)phosphine oxide, bisphenol A novolac glycidyl ether (commercial name SU-8), and the like. The viscosity of the composition can be adjusted using solvents such as γ-Butyrolactone (GBL) or propylene glycol methyl ether acetate (PGMEA), isopropyl alcohol (IPA), and the like. For example, the viscosity of the resin can be adjusted if desired to modify the shape of the tip of the structure. For example, lesser viscous liquid resin tends to form lower base angle and higher vertex angle, whereas those with higher viscosity tend to form a higher base angle and lower vertex angle. However, virtually any clear liquid resins with photoreactivity could be used, including those with added photoinitiators, such as acrylic and/or methacrylic acid esters with photoinitiator (e.g., phenones, such as benzophenones, acetophenones, as well as phosphine oxides, phospinates, etc.). Photoinitiators are commercially available including those under the Irgacure brand name, as well as triaryl sulfonium salts (e.g., Cyracure UVI, Union Carbide Corp.). Plant-based photosensitive resins are also commercially available. In some embodiments, a transparent resin is used. In some embodiments, a translucent resin can be used. In some embodiments, the resin may be opaque and be any number of available colors.

The resin is then exposed to activating radiation of appropriate wavelength and energy intensity. As shown in FIG. 1(C), the substrate 10 and resin 20 are exposed from the backside of the substrate in a “bottom-up” direction. That is, the substrate 10 with the resin layer 20 is positioned over radiation (light) source, such that the radiation source is applied to the backside of the substrate and is transmitted through the substrate from the backside to the upper surface and then into the photosensitive resin. It will be appreciated that the entire structure may be inverted and still be considered “bottom-up” so long as the radiation source is applied to the structure from the backside of the substrate as described.

Preferably, the radiation source includes a collimating lens which directs the radiation such that the direction of propagation of energy flow (light) is parallel and enters the substrate at an incident angle perpendicular to the backside surface of the substrate, and accordingly into the photosensitive resin. As the radiation passes through the open areas of the pattern, it then penetrates and propagates through the photosensitive resin layer in a direction generally away from the upper surface 12 of the substrate 10 yielding light-exposed portions and non-exposed portions of the resin layer. Inparticular, diffraction occurs to scatter the radiation intensity in such a way that the central region of radiation within each aperture has a higher intensity that gradually decreases near to the edge of the aperture. It will be appreciated that most of the resin may receive some dose of radiation exposure, such that “non-exposed portions” simply refers to those portions of the resin layer that received insufficient dosage to induce crosslinking and/or photopolymerization. The diffracted radiation polymerizes the liquid photosensitive resin once the energy of the propagated radiation in the light-exposed portions accumulates over the threshold energy of the polymerization of the photosensitive resin by the exposure time with constant radiation intensity. The light-exposed portions are crosslinked and/or photopolymerized such that the liquid-state resin is transformed into a solid-state resin structure 22 in those portions. This transition is accompanied by a change in the refractive index of the resin as it is crosslinked and/or photopolymerized. This change in the refractive index likewise modifies the propagation path of the radiation (i.e., manipulates the beam profile) as it travels deeper into the resin layer. In particular, the difference in refractive index between the solid-state resin structures 22 and surrounding liquid state resin introduces a barrier to diffraction at the interface thereby confining and guiding the direction of propagation of the radiation. As such, the resulting solid-state resin structures 22 have a generally tapered structure whereby the base of the crosslinked and/or photopolymerized structure is larger than the top of the structure (i.e., tip of the structure). Such structures are initial defined by a first height (h₁), as measured from the upper surface of the substrate to the tip.

As the radiation induces further crosslinking and/or photopolymerizing in adjacent regions of the resin layer, these crosslinked and/or photopolymerized regions thereafter act as a waveguide or lens to confine and further focus the radiation wherein the cross-section of the path of the radiation decreases with propagation distance from the light source, converging at a point (e.g., the radiation beam becomes self-focusing). The radiation exhibits a centric radiation intensity whereby the intensity is greater through the center of the aperture than at the edges, allowing it to propagate further into the resin layer. As illustrated in FIG. 1(D), the self-focusing of the radiation and subsequent crosslinking and/or photopolymerizing in adjacent regions results in further elongation of the structure tip. With additional exposure times, the resulting structures 22′ can be defined by a second height (h₂) which is greater than the first height (h₁).

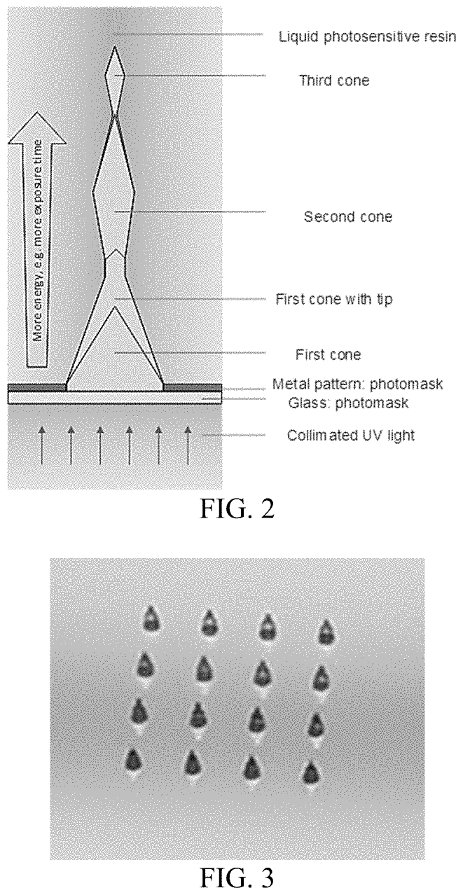

As illustrated in the working examples, the height and the shape of the cone can be changed by the applied energy in terms of radiation intensity and exposure time. More exposure creates secondary and tertiary harmonic structures as the first solidified cone shape works as meta-state light channels or lenses to create a secondary ellipsoidal shape on top of the first-formed cone, as illustrated in FIG. 2. As further illustrated in the working examples, the micro-structures may have sidewalls with varying taper angles. That is, diffraction of the radiation as it propagates through the resin can lead to sidewalls with alternating inclining and declining angles leading to more diamond shaped tips, rather than a consistent taper. The term “taper” as used herein encompasses all structures having a relative taper from a base to a pointed tip (apex or vertex), and preferably a sharp tip, where the base is wider than the tip, and is not limited to only cone or pyramidal shapes having a consistent sidewall taper. Advantageously, these complex shapes and microneedles can be formed merely by extending the exposure time and corresponding energy dosage applied to the resin, using a single exposure, which means that the exposure step does not have to be started and stopped to reposition the substrate or photomask or apply the radiation from a different angle, etc. Rather, the exposure step in the inventive method is continuously applied until the desired shape is formed.

It will be appreciated that various photosensitive resins have different crosslinking energy requirements. Further, radiation exposure tools have different light intensity capabilities. In general, applied energy or dosage (mJ/cm²) represents the most important parameter to calculating crosslinking. Dosage=intensity (mW/cm²)×time (seconds). Thus, at a higher light intensity, the exposure time can be reduced to achieve the same applied energy (dosage).

Likewise, at lower intensity, more exposure time can be used to achieve the requisite energy dosage. In general, the exposure wavelength can range from 300 nm to about 450 nm, for a time period of from about 1 second to about 1 hour, preferably from about 10 seconds to about 30 minutes. In general, applied energy dosage will range from 5 mJ/cm² to about 100,000 mJ/cm². It will also be appreciated that dosage information may be publicly available or can be determined experimentally to calibrate to fabrication process to a particular selected resin, without departing from the spirit of the invention.

After the desired structure 22′ has been formed, the structures can be developed by washing the substrate with a suitable solvent system for removing the uncrosslinked or unpolymerized resin remaining on the substrate. Suitable solvents include isopropanol (IPA), acetone, and aqueous compositions (e.g., DI water, etc.), and the like. Mechanical agitation (e.g., orbital shaking) can be used to facilitate dissolution of the unreacted resin. The substrate can then be dried yield the substrate with a plurality of micro-structures 22′ formed thereon (FIG. 1(E)).

It will be appreciated that this process facilitates formation of precise micro-structures with a single exposure step and/or single photomask. Moreover, the tapered micro-structure shape can be achieved without complicated equipment. For example, in the process, the substrate is preferably a planar substrate. Preferably, the substrate remains level during lithography or at a fixed angle. Moreover, the substrate preferably remains stationary during micro-structure lithography. That is, in preferred embodiments, it is not necessary to tilt, rotate, or otherwise move the substrate during the exposure step to form the tapered micro-structures.

The resulting micro-structures are typically characterized by a tapered shaft. More preferably, the width or diameter of the micro-structures is greatest at the base end of the micro-structure adjacent to the substrate, and tapers to a point at the end distal the base. Depending upon the shape of the pattern used to form the structures, the micro-structures can be formed with shafts having circular cross-sectional geometries (cone-shaped), or any other desired shape, including square base (pyramidal), star-shaped, triangular, oblong, etc. The angle of the taper can likewise be varied. As noted in the compression testing below, the steeper the angle, the sharper the tip. Depending upon the intended use, the sharpness of the tip can be balanced with the strength of the structure. Typically, if the apex angle is too small (e.g.,)≤30°, the micro-structure can easily break upon application of force. However, it will also be appreciated that the break point is also dictated by the particular material used to fabricate the micro-structure as well as the overall size/width of the tip. In one or more embodiments, the process can be used to fabricate structures with a size (edge-to-edge dimension, i.e., diameter), as measured at the base, of from about 5 μm to about 1,000 μm, more preferably from about 50 μm to about 300 μm. The height of the micro-structures, as measured from the substrate surface to the tip can range from about 30 μm to about 9 mm, more preferably from about 300 μm to about 1,000 μm. Exposure times typically are less than 1 hour, even more preferably less than 45 minutes, and even more preferably less than 30 minutes.

Moreover, as also exemplified in the working examples, this process can be further modified to achieve the desired micro-structure shape. For example, hollow micro-structures can be formed by using a pattern where the apertures have a solid core to block transmission of light, and accordingly block crosslinking and/or photopolymerization in this central region. As such, the radiation is transmitted through an annular ring in the mask, and solidifies a corresponding portion of the resin adjacent to the mask pattern. Upon removal of the non-crosslinked or non-polymerized resin, the resulting structures are hollow with a substantially annular bore or channel extending from the base of the structure to the tip. The micro-structures can be fabricated to extend away from the substrate upper surface 12 in a substantially perpendicular direction. Alternatively, as also demonstrated in the examples, they may be fabricated at an angle to the substrate surface 12. Depending upon the pattern used, an array of micro-structures can be fabricated across a substrate in a single exposure process having a mixture of different geometries and/or sizes and/or angles by using a pattern having different sized and/or shaped apertures.

Additional modifications include the application of one or more intervening layers adjacent the substrate upper surface 12 before the photosensitive resin is applied. Such intervening layers can facilitate lift-off of the micro-structures. The intervening layers may also be used to further refine the direction of propagation of activating radiation into the resin layer or the pattern used to block transmission. Intervening layers may be rigid or flexible. A shadow mask is exemplified in the examples as but one example of such intervening layers. For example, an intervening layer can be applied to the substrate wherein the intervening layer has (e.g., pre-formed, or may be patterned in situ to have) corresponding aperture which align with the aperture array on the substrate. In the case of hollow micro-structures, the intervening layer can secure or stabilize the formation of the inner sidewall and hollow bore through the micro-structure during diffraction lithography, and ensure that the hollow bore extends from the base of the structure through the tip. The intervening layer in this embodiment also facilitates lift-off of the patterned microstructure array after lithography and development, as it stabilizes the structures formed. In a further embodiment, an intervening layer can be applied to the substrate as a planarizing layer. That is, although planar substrates are exemplified herein, the substrate surface may be non-planar with one or more height variations across the substrate surface. Moreover, the photomask may be itself an intervening layer with a non-planar surface (e.g., open holes and solid portions). It will be appreciated that certain modifications to the substrate surface structures or mask may be used to change the characteristics of the micro-structures to be formed by altering the path of light during the exposure process. Thus, an intervening layer can be applied to the substrate surface (or photomask) to first planarize the layer before applying the photosensitive resin.

It will also be appreciated that the resulting micro-structures can be used as a template for traditional micromolding to further fabricate additional micro-array structures using non-lithography techniques. For example, the substrate and micro-structures can be used to create polydimethylsiloxane (PDMS) molds (negatives), which can then be used to fabricate the microstructures using a variety of non-photosensitive polymer compositions using imprint molding. In this embodiment, the PDMS is applied over the diffraction lithography-formed micro-structures and cured to create a negative mold. This negative mold can then be used to form corresponding micro-structure arrays using various polymers (e.g., non-photosensitive resins) by applying a liquid resin over the negative mold, curing the resin, and peeling away the PDMS mold. It will be appreciated that the subsequent micromolding process option expands the possible resin systems from which the micro-structures can be fabricated, such that the resulting structures are not limited to photosensitive resins. For example, microneedles can then be fabricated using micromolding from a variety of biodegradable materials (e.g., for coated and/or dissolving microneedles), as well as from various hydrogels.

The resulting micro-structures formed via diffraction-lithography (or subsequent micromolding) have a variety of potential applications, including as microneedles for both medical/clinical and cosmetic uses, microprobes for electrical signal stimulation or detection, as well as for light stimulation or detection. Using the same principles leveraged for their fabrication, the micro-structures can be used as light waveguides and the light transmitted through the microcone will emit at the tip of the microcone.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Introduction

This work describes a self-focusing, diffraction ultra-violet (UV) lithography method for fabricating various micro-cone shape microneedle structures. The general process is illustrated in FIG. 1. A direct UV exposure with to a liquid state photosensitive resin through a photomask generates a unique diffraction pattern of the light, in which the exposed area of the photosensitive resin becomes the needle structures. The change of liquid to solid-state of the resin by photopolymerization and/or crosslinking changes the refractive index of the photosensitive resin, such that the photopolymerized resin acts as a light waveguide to direct and focus the light as it propagates through the resin to create a novel sharp tip on top of the cone. Removal of uncured resin leaves behind microcone shapes. In more detail, collimated light is used, which propagates like a plane wave and is diffracted upon reaching the apertures in the photomask. The diffracted light intensity distribution at the opposite side of the apertures behaves like a Gaussian profile, also known as Airy disk, which has high intensity at the center of the aperture and low at the peripheral. Liquid state photosensitive resin is crosslinked/solidified by the diffracted light, forming a small cone with the Gaussian profile on the aperture. The solidified resin has a higher refractive index than the surrounding uncrosslinked liquid resin, such that light that propagates through the solid resin is refracted at a wider angle to the incident angle as it passes through the solid-liquid interface (which defines the sidewall of the micro-cone structure), and may even be reflected back off of the interface into a conical light profile. This sidewall of the micro-cone structure acts like a waveguide and focuses all of the light to one point resulting in a centric light intensity causing the cone angle to become steeper and eventually forming the needle-shaped cone tip (first harmonic).

As illustrated in FIG. 2, further UV energy exposure can lead to elongation of the cone tip, and the formation of secondary and tertiary harmonic cone shapes with different geometric profiles. In particular, once the first harmonic is formed, the sharp tip of the first harmonic acts like a secondary light-focusing aperture, such that a secondary cone structure can be formed via the stronger light intensity at the center/tip of the forming structure. The shape of the microneedle at this point is essentially an optimal shape which has a slightly tapered body and isosceles triangular tip with a taper angle of approximately 30° . With further exposure energy applied, the secondary harmonic structure will be formed, followed by the formation of the third core and tertiary harmonic.

The proposed method is unique and versatile as it enables the formation of various cone-shaped microstructures with straight, angled, or curved sidewalls, such as tip-integrated cone and multiple harmonic cones having different heights and base shapes, as well as microneedles with the optimal shape, standard tapered needle structure, or even micro-cone structure with a rounded tip by single backlight exposure on one photomask. Microneedle height and shape can be modulated by using different exposure energy and resin materials. A photograph of a resulting UV diffraction experimental validation using a 4×4, 200-μm photomask pattern is shown in FIG. 3, where the formation of microcones on the surface of the substrate is clearly discernible.

### Example 1

In the initial experimental set-up, a glass slide with a photomask having a photopattern of several openings was coated with a photosensitive resin. A UV-LED covered with a collimated lens was located underneath the photomask. The liquid photosensitive resin was poured on top of the photomask until it covered the photomask surface, but was held in place via surface tension. The selectable wavelength of the UV LED in a range of 300 nm to 450 nm is applicable for this fabrication. Wavelengths of 365nm, 375 nm, 385 nm, 395 nm, and 405 nm peak LEDs have been tested and verified to form the microcones. Each wavelength has different optical characteristics to the photosensitive resin including transparency and attenuation behaviors.

A clear resin from Anycubic POT016 LCD UV 405 nm Rapid Resin was used for one fabrication, with a photomask array with 200 μm apertures. The thickness of the photosensitive resin on the photomask was around 2 mm, which is thicker than the target height of the microcones. The light exposure time varied from 10 seconds to 30 minutes at the UV intensity of 10 mW/cm², depending on the targeted cone profile. After exposure, the sample was soaked in isopropyl alcohol (IPA) for 10 minutes on an orbital shaker at 20 rpm to remove uncured resin. After the developing process with the IPA, the sample was dried to complete the process.

TABLE 1
Optical dose* for different microcone profiles

| Time in sec | Height (mm) | Tip | Length of tip | Width of tip |
|---|---|---|---|---|
| 5 | 0.407 | no | | |
| 10 | 0.594 | no | | |
| 30 | 0.739 | no | no | no |
| 65 | 0.808 | about to form | no | no |
| 120 | 0.736 | yes | 0.135 | |
| 150 | 0.87 | yes | 0.212 | 0.067 |
| 180 | 0.9 | yes | 0.286 | |
| 210 | 0.893 | yes | 0.259 | 0.076 |
| 240 | 0.852 | yes | 0.264 | |
| 300 | 1.047 | yes | 0.346 | 0.072 |
| 360 | 0.98 | yes | 0.327 | 0.091 |
| 450 | 0.989 | yes | 0.322 | 0.092 |

*Each dose at 365 nm wavelength through 200 μm circular mask, intensity 10 mW/cm²

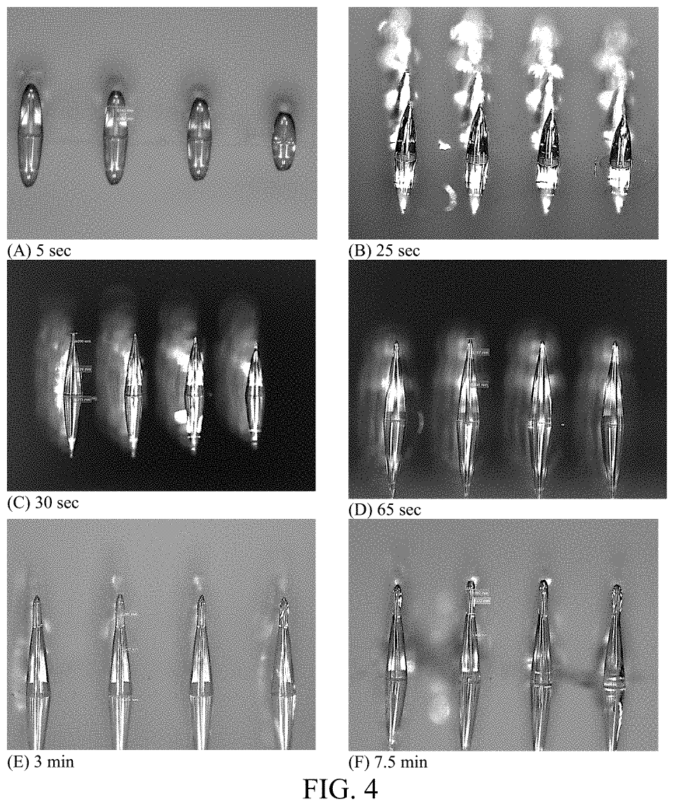

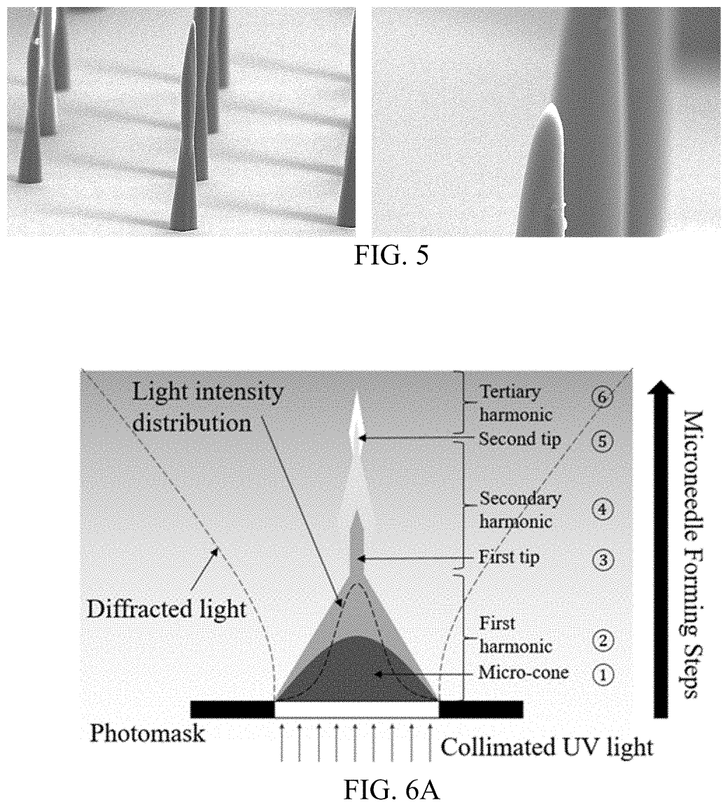

Photographs of the various microstructures formed at different exposure times are shown in FIG. 4. As shown in the images in FIGS. 5(A)-(B), the initial experiments were able to also fabricate secondary harmonic cones through additional exposure times. These experiments were carried out using a 120 μm photomask.

### Example 2: Diffraction Lithography for 3D Microneedle Fabrication

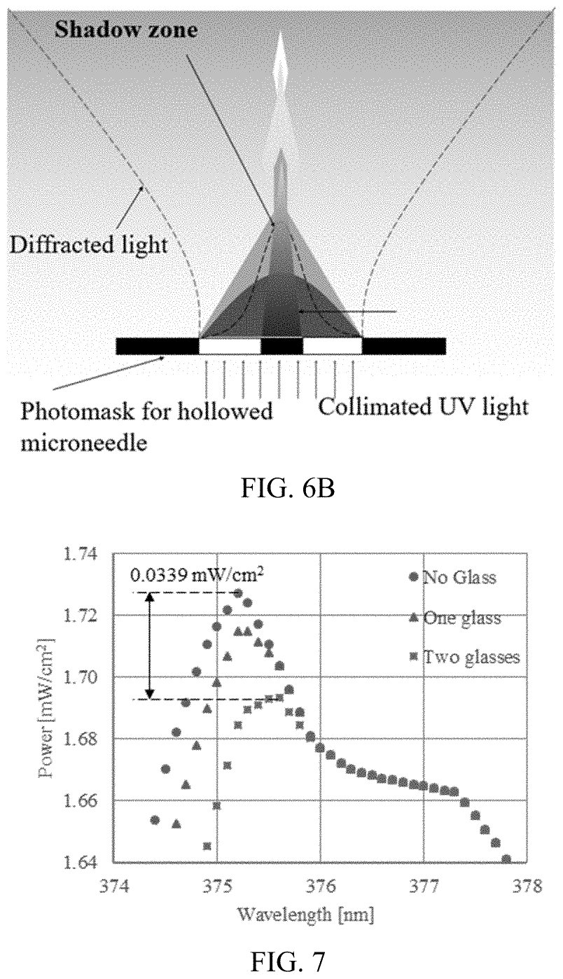

Strategies for formation of solid and hollow microstructures, and subsequent building of different geometries are illustrated in FIG. 6A and FIG. 6B, and rely on principles of light diffraction and intensity distribution, as well as the variability of the refractive index of the photosensitive resin as it changes from a liquid state to a photopolymerized and/or crosslinked/cured solid state. As illustrated in FIG. 6A, a “bottom-up” exposure process is used with collimated light, where (1) an initial micro-cone structure is formed as a base structure adjacent the substrate upon exposure to the light source through the photomask aperture. In (2), the micro-cone sidewall, which has formed as the liquid resin changes to a solid state now acts as a waveguide to direct the light to form first harmonic. In (3), the first cone tip formed as the light which propagates through the resin self-focuses into a conical light profile such that it the light intensity converges at the tip. In (4), as more energy is applied and focused at the tip to photopolymerize and/or crosslink/cure the resin in this area, the secondary harmonic structure is thus formed. Again, the secondary harmonic structure likewise focuses the light intensity to form the second sharp tip (5), and a tertiary harmonic can be similarly formed as even more energy is applied (6).

As illustrated in FIG. 6B, a similar technique can be used to form hollow micro structure, except that the photomask is further patterned so that a central region of the aperture is blocked to prevent light from passing through that portion of the aperture, thus creating a shadow zone in the central region of the forming structures where the resin remains uncured. As such, the central core of the structure can be hollowed out by removing the uncured resin after patterning.

Using these principles, several different microstructures were formed. In one experiment, a glass slide was used as the transparent substrate, onto which a patterned photomask was placed. The photomask was covered with the photosensitive resin. In these experiments, UV LEDs having various wavelengths (365, 375 and 405 nm) integrated with a collimation waveguide were set as the light sources. Different wavelengths will have a different attenuation/absorption rate inside the liquid photosensitive resin. Longer wavelengths will have lower attenuation and therefore may result in taller structures. In general, the thickness of the liquid photosensitive resin should be thicker than the desired height of the structures to be fabricated.

A photomask with an array of circular apertures was coated with clear photosensitive resin (Clear Resin, Formlabs Inc.) and placed on a microscope glass slide located 13 mm above the light source. Considering that in this setup, the light has to pass through two slides of glass (microscope glass slide and photomask glass) before it reaches the resin, the optical energy was measured with a spectrometer (BLUE-Wave Miniature Spectrometer, StellarNet Inc.) at a constant distance of 13 mm above the light source. The results in FIG. 7 show that the measured peak optical energy was 1.7271 mW/cm² when there was no glass in the optical path, 1.7149 mW/cm² when there was one glass and 1.6932 mW/cm² when two glasses were in the optical path. The optical energy was attenuated by 0.0339 mW/cm² while passing through two slides of glass, which was also equivalent to 2% of attenuation that is negligible in this experiment.

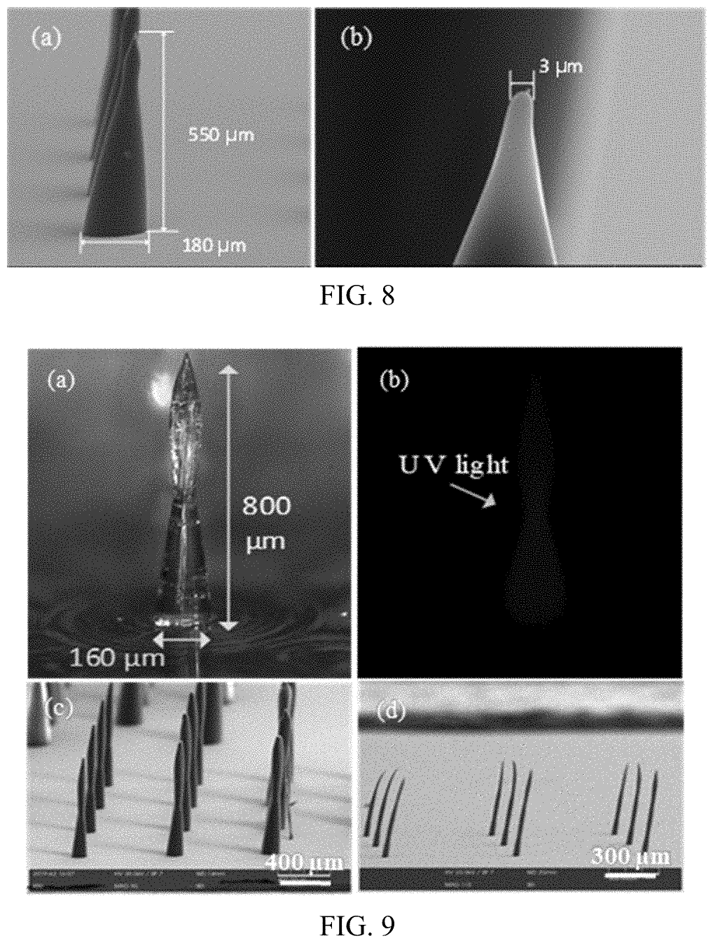

Microstructures were patterned by exposing the photosensitive resin with direct backlight 375 nm UV light source. The slide was then transferred into isopropanol (IPA) to remove the uncrosslinked resin with slight orbital shaking (20 rpm). Once the development was completed, the sample was gently dried with compressed air and the microneedles with the tapered body were completed. The SEM images (FIG. 8(A)-(B)) show that an array of microneedles was successfully fabricated on a circular aperture patterned photomask with single backlight UV exposure. The optimal tip shape of microneedles with a taper angle of around 30° was achieved. The microneedles had a measured base diameter of 180 p.m and a height of 550 p.m, with an aspect ratio of 3.06. The tip diameter was 3μm with a taper angle of 25.7° , which is sharp enough to penetrate the skin without fracture failure.

In another experiment, more exposure energy was applied on another photomask with a smaller circular aperture, and 800 p.m microneedles with a second harmonic structure were successfully fabricated as shown in FIG. 9(a). The base diameter and height of the microneedles were 160 p.m and 800 p.m, respectively, with an aspect ratio of 5. In FIG. 9(b), an optical image of UV light captured during the fabrication process shows the propagation of the light inside a liquid photosensitive resin and experimentally verifies that a second harmonic was achievable with sufficiently applied energy. An array of microneedles with secondary and tertiary harmonic is shown in FIGS. 9(c) and 9(d), demonstrating the manufacturing capability of the proposed method as well as its uniqueness in fabricating complex microneedle structure with only single UV exposure.

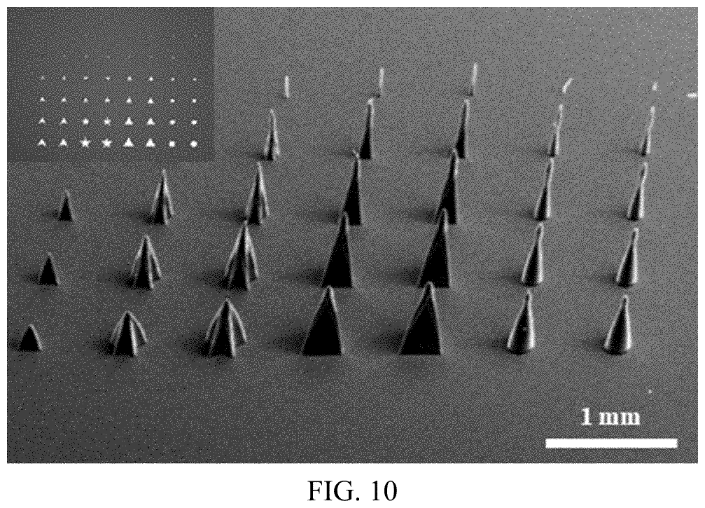

To further demonstrate this versatile fabrication method, various photomask aperture geometries (circular, triangle, star and triangle with curved base) with various aperture sizes were used to fabricate microneedles. FIG. 10 shows the different aperture geometries (inset) used to generate different optical diffractions which form various microneedle shapes on the same substrate with single UV exposure.

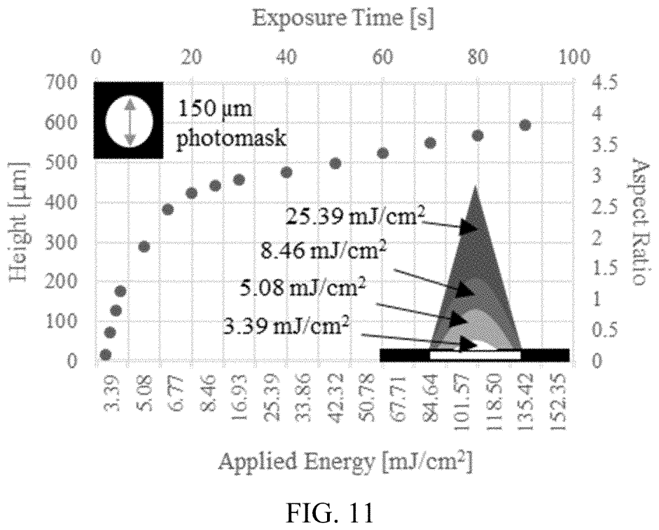

To study the minimum amount of energy required to initiate crosslinking of the liquid photosensitive resin, a photomask with circular aperture size of 150 p.m (inset) and optical energy of 1.6932 mW/cm² by 375 nm UV light source was set as constants while the exposure time was set as a variable (1 sec to 90 sec). The data (FIG. 11) shows the relationship between the fabricated microneedle height and the energy applied to crosslink the resin. The secondary y-axis shows the aspect ratio of the corresponding height and the secondary x-axis shows the exposure time of the corresponding energy applied. The minimum amount of energy to crosslink the photosensitive resin was 3.39 mJ/cm² and the measured height was 8.4 μm. The micro-cone structure grows relatively fast during the first 20 seconds, and a constant growth rate of 2.44 μm for every 1 mJ/cm² of additional energy application was observed afterward.

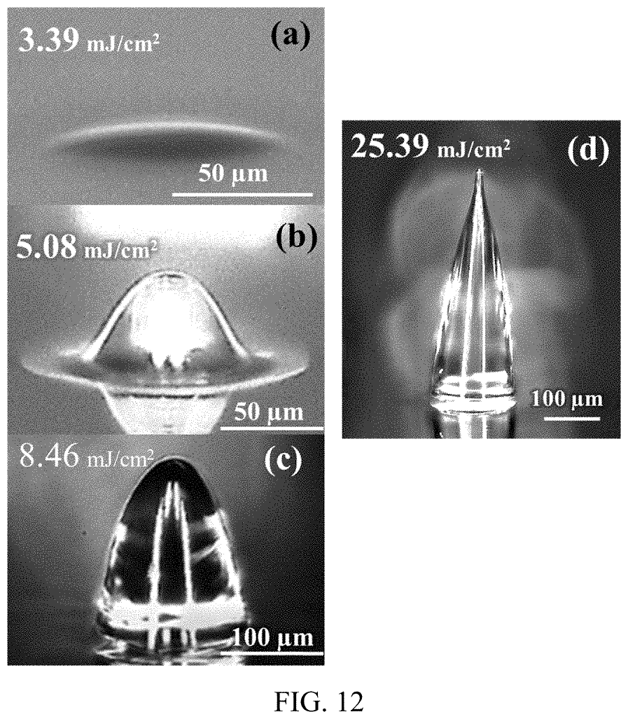

Optical images of micro-cone structures at 2, 3, 5 and 20 seconds of exposure time are shown in FIG. 12, where microneedle sharp tip characteristic begins to exhibit at 20 seconds exposure time.

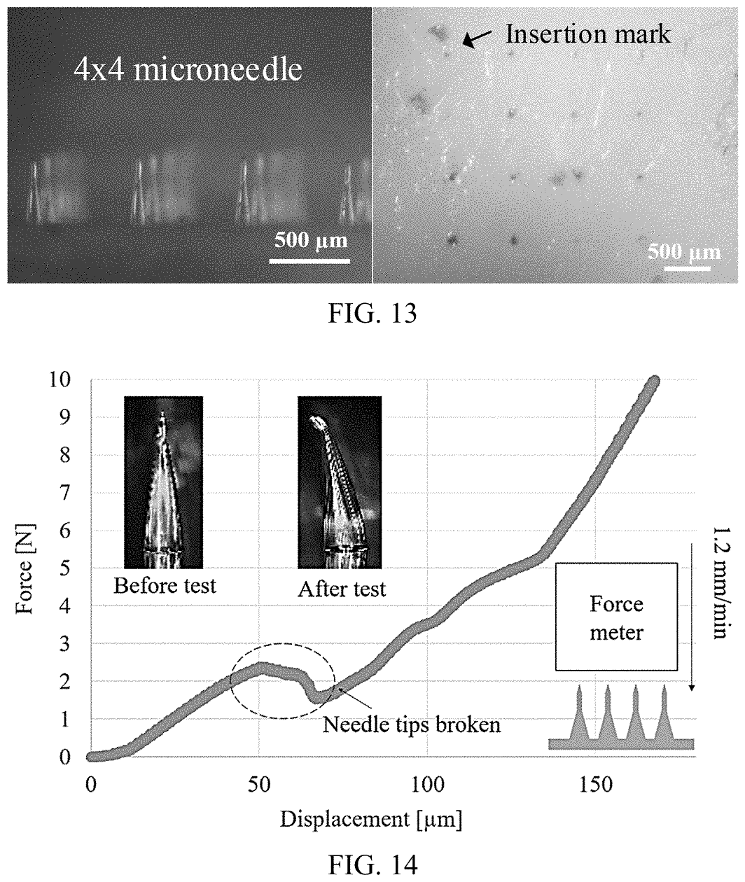

Insertion tests and force-displacement tests were conducted to demonstrate the functionality of the fabricated microneedle, and the results showed that the tip of each tested microneedle could withstand up to 0.15 N before breakage occurs. The fabricated cone-shaped microneedle with a sharp tip has a great potential for transdermal drug delivery microneedle application. To prove the functionality of the fabricated microneedles, a 4×4 microneedle array with the same overall geometry shown in FIG. 8 was prepared from polylactic acid (PLA) by micromolding. This geometry was selected for the mechanical test as it is similar to other microneedle geometries that have been reported to penetrate the skin without failure. PLA microneedle array shown in FIG. 13(a) was inserted into pig cadaver skin by thumb pressure, and the insertion area was stained with blue tissue marking dye for visualization.

FIG. 13(b) shows 4×4 insertion marks in blue on the pigskin. A force-displacement test was also conducted using a 4×4 microneedle array. The microneedles were designed based on

FIG. 11 to have a diameter of 150 μ.m and a height of 500 μm, where the tip was 80 μm long with a taper angle of 27.6° . A force gauge (FC200, Torbal Inc.) was installed on a stepper motor integrated with a threaded rod along the z-axis and controlled by Arduino (Arduino UNO Rev 3, Arduino). The 4×4 microneedle array was placed directly under the force gauge and the force gauge was commanded to move down at a speed of 1.2 mm/min while recording the force every 1 millisecond. The total test time was converted into displacement in micrometer and the result was plotted (FIG. 14). As the microneedle was compressed, the first tip began to deform at 2.38 N, indicating that each needle tip can withstand at least 0.15 N without any mechanical failures. After that, tips were bent, resulting in a sudden drop in force. Once all the tips were completely deformed, the detected force increased linearly, indicating that the microneedle bodies still remained attached to the substrate without deformation, which is also shown in the corresponding image. This characteristic shows great availability for the drug transportation method such as ‘coat and poke’ and ‘poke and release’. In other words, the microneedle tips can be pre-coated with drug or be made out of drug itself and could be designed to be broken during insertion while the needle bodies serve as delivery support that can be disposed after use.

The fabrication method has demonstrated its uniqueness in forming micro-cone shaped microneedle structures by light diffraction and the corresponding intensity distribution. With a simple system setup of LEDs with light collimation, microneedle arrays with complex geometries can be fabricated by only single exposure within 30 minutes. Insertion test and force-displacement test were conducted, and the results demonstrated that the fabricated microneedle tip can withstand up to 0.15 N before deformation occurs, which is durable enough for skin penetration. The fabricated cone-shaped microneedle with a sharp tip has a great potential for transdermal drug delivery microneedle application.

Example 3: Fabrication and characterization of nipple tip, hollow, and inclined microneedles by UV-LED lithography

The proposed fabrication method has advanced on top of the prior work as it introduces the functional microneedles like hollow and inclined microneedles with a single UV exposure scheme. The hollow needles are useful for liquid state drug delivery, and the inclined microneedle can be applicable to non-planar skin surface working as a hook. And those microneedle fabrication processes can be done within 30 minutes including sample preparation,

UV exposure, developing, and cleaning which will significantly reduce the production cost. Tests were carried out with various wavelengths of UV LEDs (365, 375, 385, 395 and 405 nm) to generate different shape of the microneedle.

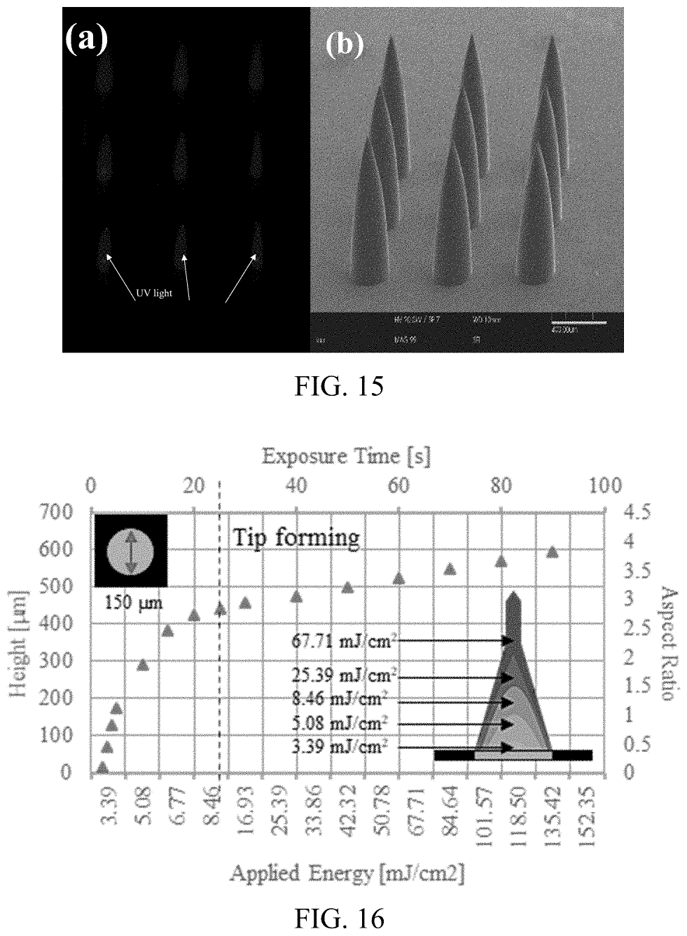

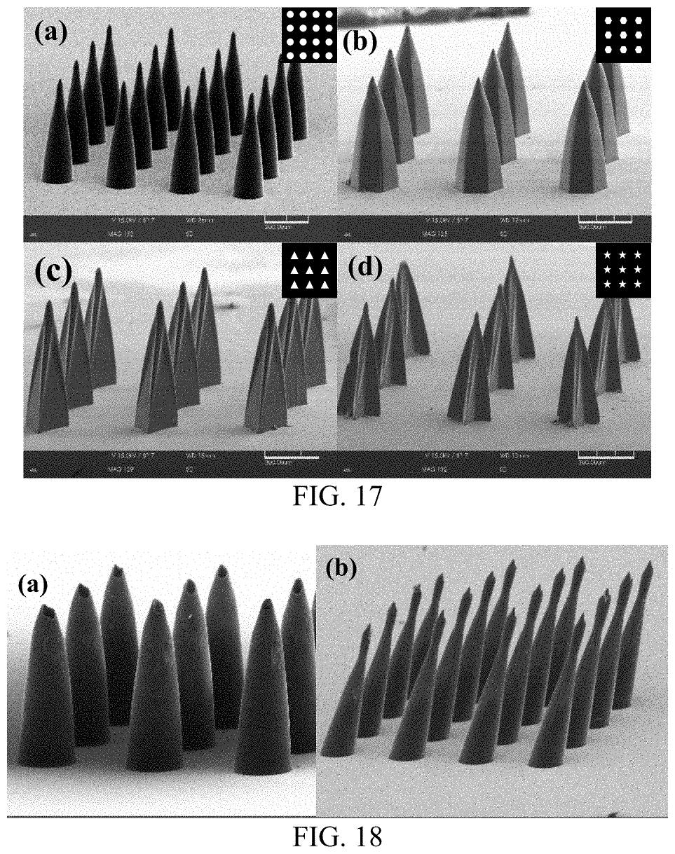

As shorter wavelength gets attenuated more than the longer ones, the shape prediction of the microneedle can be investigated from the experimental setup. FIG. 15 shows the UV light propagation test inside the photosensitive resin. FIG. 15(a) shows the shape of the light propagation, and FIG. 15(b) shows the SEM picture of the corresponding 3×3 circular microneedle array. The data in FIG. 16 shows the relationship between the microneedle heights at different applied energy with a 150-μm photomask, and the drawing shows the general needle shape at corresponding applied energy. Using this data and various photomask geometries, different microneedles can be formed, as shown in FIG. 17. The fabrication method was also used to create hollow microneedles and inclined circular microneedles as shown in FIG. 18.

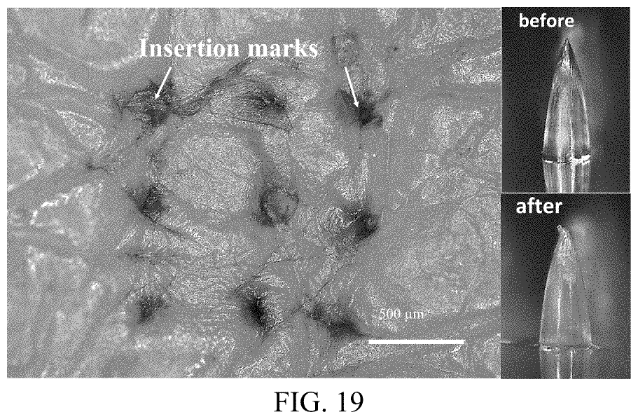

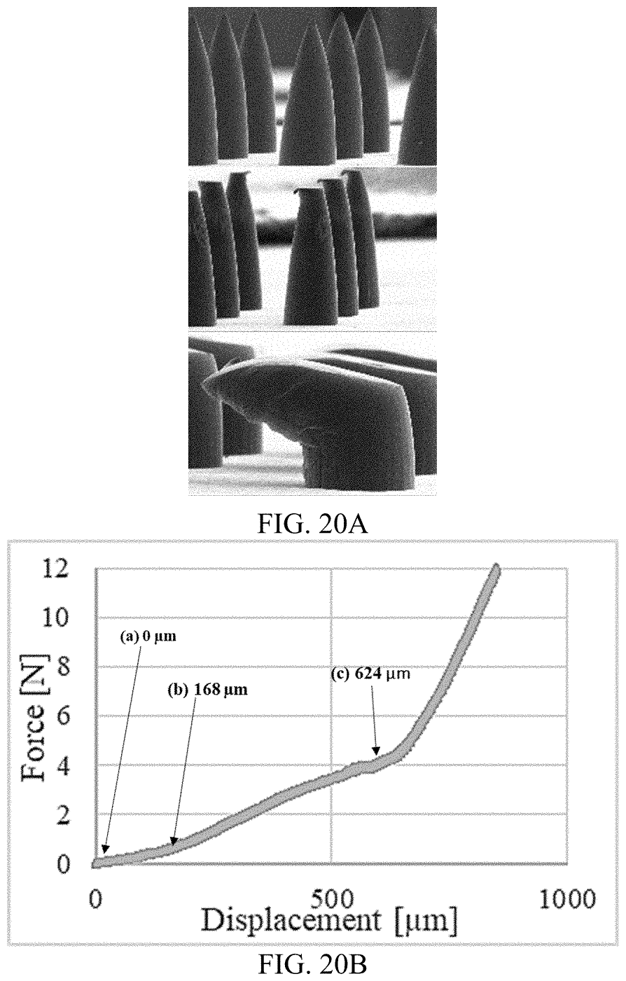

Insertion and force-displacement experiments were also conducted using a 3×3 PLA circular microneedle array. Insertion testing on pig skin with blue dye markings visualize the needle insertion spots on the pigskin (FIG. 19). Force-displacement data is shown in FIGS. 20A and 20B. SEM images are shown in FIG. 20A for a 3x3 PLA circular microneedle array with a height of 750 p.m fabricated based on the data from FIG. 16. The microneedles were placed underneath the force meter and the compression speed of the force meter was set at 1.2 mm/min. The top panel shows the microneedles before any displacement. The middle pane shows the shape of the microneedle after 168 μm of displacement. Only the needle tip started to deform while the needle body remained unchanged after the compression. The bottom panel shows the shape of the microneedle after 624 μm of displacement that most of the needle body was deformed or bent, while the base remains relatively in place. As shown in FIG. 20B, the force-displacement slope also drastically increased at this point, which implies that the durability of the microneedle body is qualified to use for microneedle application.

Example 4: Advanced Microneedle Fabrication

In this work, microneedles were fabricated using a Surgical Guide Resin from Formlabs (Somerville, Mass.). This resin is a commercially-available, autoclavable, biocompatible resin typically used for 3D printing of dental surgical guides for implant placement. The resin is a trade secret formulation from the company comprised of methacrylate monomer (25-45 wt %), urethane dimethacrylate (55-75 wt %), and photoinitiator (1-2 wt%) according to the MSDS.

### I. Minimum Crosslinking Energy

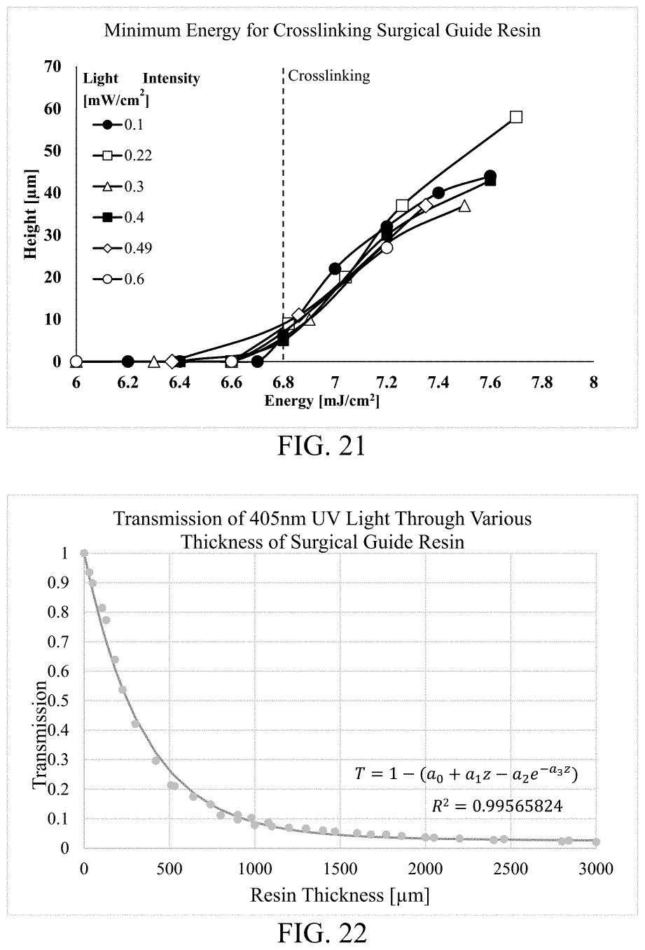

Initial testing investigated the minimum amount of energy required to crosslink surgical guide resin. Understanding the crosslink energy allows predicting the crosslink height more accurately. A 405 nm UVLED (UV 405 nm LED, Shenzhen Chanzon Technology Co., Ltd.) was used as the UV source. A 3D printed waveguide was used with the UVLED for light collimation. A plain glass slide was positioned above the waveguide with a constant distance of 1 mm to minimize the loss of UV intensity through space. A thin layer of surgical guide resin was spin-coated onto the plain glass at a thickness of 50 μm. Lastly, a UV intensity meter was set at 1 mm above the surface of the resin to monitor the intensity of the UV source. Very low UV light intensity was applied in increasing increments (0.1, 0.22, 0.3, 0.4, 0.49, and 0.6 mW/cm²) in order to precisely measure the crosslinking timing. The results are in FIG. 21, and show the resulting height of the crosslinked resin at various light intensities and various exposure energy in an incremental fashion. Regardless of light intensity, for this resin, when the exposure energy reaches 6.8 mJ/cm², the height of the crosslinked resin increases, indicating that the minimum crosslinking energy for this surgical guide resin is 6.8 mJ/cm².

II. Transmission rate of 405 nm UV light through various thickness of surgical guide resin.

The transmission rate of 405 nm UV light through various thickness of surgical guide resin was investigated. Understanding the transmission rate of 405 nm UV light also helps better prediction for the crosslinking be havior of the surgical guide resin. A 405 nm UVLED (UV 405 nm LED, Shenzhen Chanzon Technology Co., Ltd.) was used as the UV source. A 3D printed waveguide was used with the UVLED for light collimation. A plain glass slide was positioned above the waveguide with a constant distance of 1 mm to minimize the loss of UV intensity through space. Unlike the previous section, various thickness of surgical guide resin was applied on the plain glass, which varies from 0 to 3000 μm. Lastly, a UV intensity meter was set at a constant distance of 11 mm above the plain glass to monitor the intensity of the UV source for various thickness of surgical guide resin. Before the surgical guide resin was applied, the UV light intensity was measured and recorded as the initial UV intensity, I₀. The UV light intensity was again measured after the resin was applied and referred as parameter “I”. Using the two recorded intensity, the transmission rate was calculated using the equation below:

$$Transmission = T = \frac{I}{{I}_{0}}$$

The results are in FIG. 22. A fit curve was generated as shown in the equation below, giving an R² of 0.99565824. The attenuation factor, a₃ for the surgical guide resin was 0.00287837.

$$Transmission = T = \frac{I}{{I}_{0}} = 1 - ( {a}_{0} - {a}_{1} z - {a}_{2} {e}^{- {a}_{3} z} ) \qquad ( 1 )$$

where α₀=0.96265990

α₁=0.00000366

α₂=0.96265990

α₃=0.00287837 (Attenuation factor)

To predict the crosslinked resin height, we start with a basic formula to calculate energy based on the UV light intensity and exposure time:

Energy=E=I·t (2)

where I is the 405 nm UV light intensity in mW/cm² and t is the exposure time in seconds. Based on Equation (1), we know that I is a function of z, where z is the thickness of the surgical guide resin, therefore, we can re-write Equation (1) as:

I=I₀−I₀(a₀+a₁z−a₂e^{-a}^{₃}^{z})

Then, substitute into Equation (2):

E=I₀t−I₀t(a₀+a₁z−a₂e^{-a}^{₂}^{z})

where I₀ is the UV light intensity when z=0. Based on Equation (3), if we fix I₀ and t, we can see that E is inversely proportional to z, the higher the thickness of resin, z, the lower the received energy, E. Knowing this relationship, we can say that, at every constant value of I₀ and t, there must be a vertical thickness of resin, z that corresponds to the minimum crosslinking energy of surgical guide resin, which is 6.8 mJ/cm² as we have measured and discussed above.

To verify the equation, a realistic set of data was generated using conditions as shown in below:

| | Parameters | Values | Units |
|---|---|---|---|
| Voltage | V | 2.88 | V |
| Current | I | 14 | mA |
| Power | P | 40.32 | mW |
| UV Wavelength | λ | 405 | nm |
| Optical Intensity at z = 0 | I₀ | 5.75 | mW/cm² |
| Exposure time | t | 0 to 900 | Seconds |
| Energy | E | 0 to 5175 | mJ/cm² |

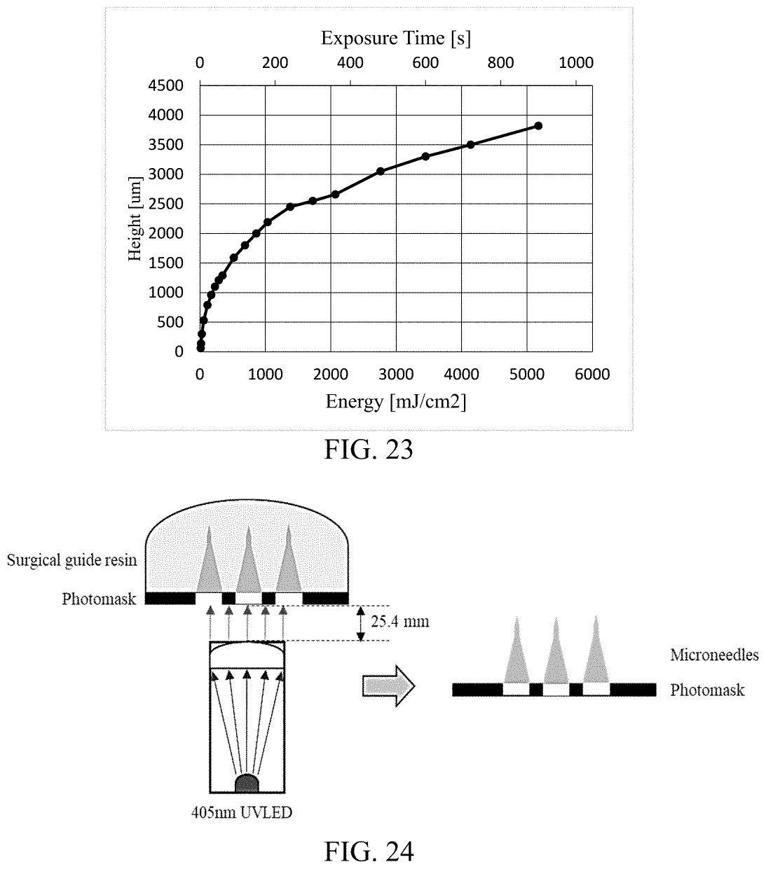

Using the same experiment system setup and the experiment conditions listed above, the heights of the crosslinked resin at various amount of energy were measured and recorded as shown in FIG. 23.

### III. Microneedle Height Characteristics

Next, the characteristics of the microneedle and its height at various exposure energy was investigated. The conditions of this experiment are listed in the table below:

| | Parameters | Values | Units |
|---|---|---|---|
| Voltage | V | 3 | V |
| Current | I | 60 | mA |
| Power | P | 180 | mW |
| UV Wavelength | λ | 405 | nm |
| Optical Intensity at z = 0 | I₀ | 19.65 | mW/cm² |
| Exposure time | t | 0 to 120 | Seconds |
| Energy | E | 0 to 2358 | mJ/cm² |

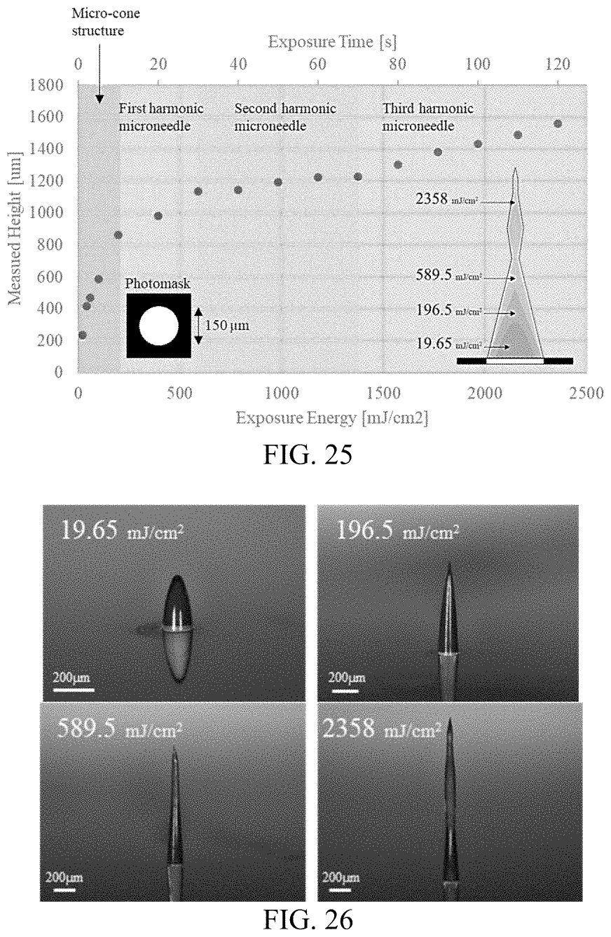

FIG. 24 illustrates the system setup for microneedle fabrication for these experiments. Starting from the bottom, a waveguide-integrated 405 nm UVLED was used as the UV light source, where a collimated lens transforms the LED light into parallel light. A patterned photomask with 150 μm aperture pattern was set at a distance of 25.4 mm from the UV light source, which serves as the substrate for surgical guide resin. A layer of the surgical guide resin was applied on the photomask and then exposed with 405 nm UV light. The exposure process was stopped when certain exposure energy was reached, and the corresponding microneedle heights were recorded at those designated exposure energy points. Once the exposure process was complete, the sample was washed with isopropanol and the microneedles fabrication is complete. FIG. 25 shows the measured heights of the microneedles at various exposure energy. In this particular example, the graph is separated into four sections based on the shape of the microneedle structure including micro-cone structure, first harmonic microneedle, second harmonic microneedle, and third harmonic microneedle (FIG. 25). FIG. 26 shows photographs of micro-cone structure and microneedles that represent the shape at each stage of exposure energy. This finding shows that various sized and shaped microneedles can be fabricated based using a single photomask and simply varying the exposure energy.

### IV. Microneedle Array Fabrication

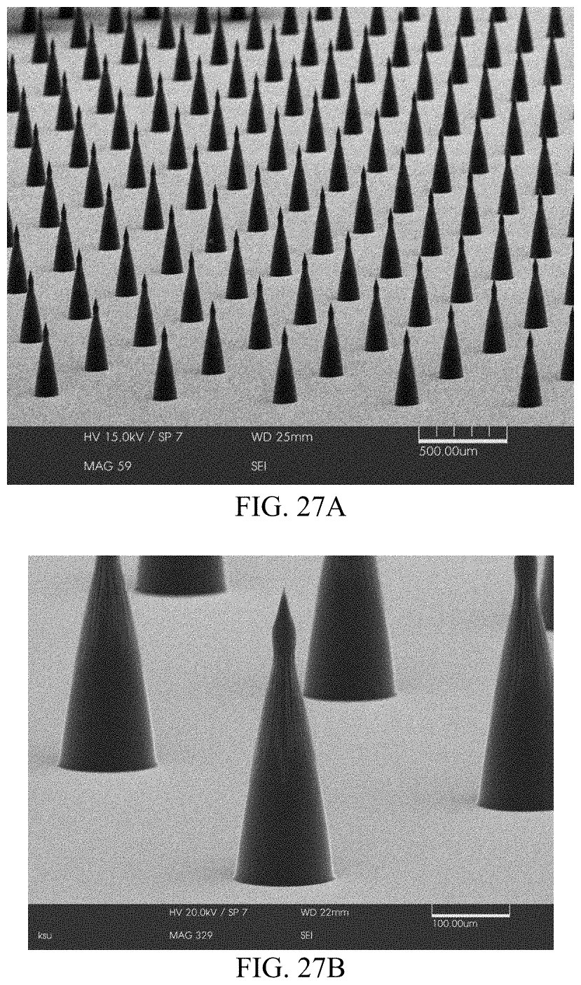

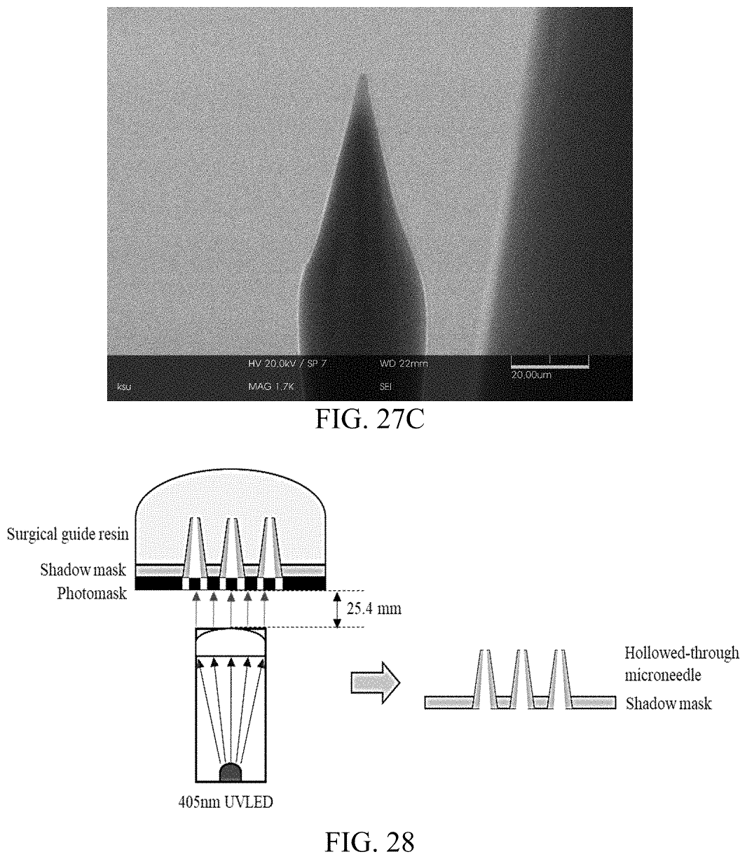

Using the above set-up (FIG. 24), various microneedle arrays were fabricated using the surgical guide resin. A photomask with a 20×20 array of apertures having 150 μm diameter openings was used to fabricate solid straight microneedles. FIG. 27A shows an SEM image of the resulting array after development of the substrate and removal of uncured resin. As can be seen, the process results in a 20×20 array of microneedles with consistent size and shape, each having a base diameter of 133 μm with an average height of 385 μm. Enlarged SEM images of an individual microneedle (FIG. 27B) and of an individual tip (FIG. 27C) are also provided. The microneedle tip had an approximate width of 2.5 μm and a tapered angle of 28.5°.

Using a modified set-up shown in FIG. 28, hollowed-through microneedles were fabricated.

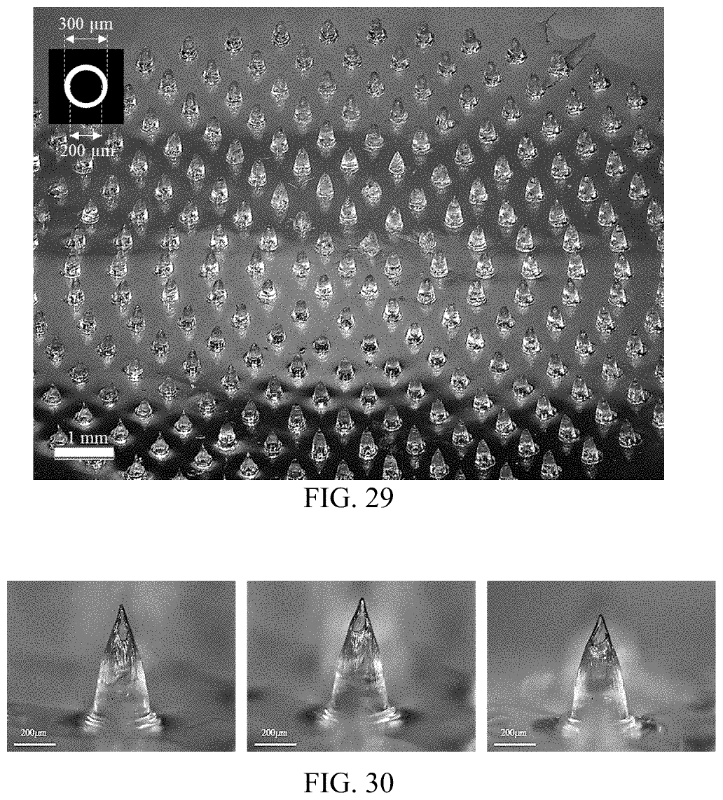

As with the other experiments, a waveguide-integrated 405nm UVLED was used as the UV light source. A patterned photomask was set at a distance of 25.4 mm from the UV light source. The photomask array included 271 apertures each with a solid core to prevent transmission of light in the center of the apertures (see inset image in FIG. 29). The apertures had an outer diameter of 300 μm, with a solid core having a diameter of 200 μm, thus leaving a 100 μm-wide annular ring open for light transmission. To further enhance the effect, a shadow mask (made with resin) with complete through holes was prepared using 3D printing, and aligned with the photomask apertures using a mask aligner. The shadow mask that was placed on top of the photomask contains 271 units of complete through holes with a diameter of 300 μm (open core) complementary to the photomask apertures. Once the UV exposure process was completed, the sample was washed with isopropanol and the shadow mask was detached from the photomask along with the hollowed-through microneedles. A photograph of the resulting 271 units is in FIG. 29. Since the material that forms the hollowed-through microneedles is the same as the shadow mask, the shadow mask can be easily detached from the photomask along with the hollowed-through microneedles as the bonding to the shadow mask is stronger than the photomask. The hollowed-through microneedles were measured to have a base diameter of 280 μm and a height of 550 μm (FIG. 30, enlarged view). As an be seen in the views in FIG. 30, the individual needle shapes and sizes are consistent across multiple needles.

### V. Insertion Test

A pig skin was first cleaned with isopropanol alcohol to remove possible contamination. A 20×20 solid straight microneedles array was fabricated using diffraction lithography and then used as a template to create a PLA array using a PDMS micromolding process. Briefly, after fabricating the diffraction lithography microneedle array on a glass substrate, a PDMS process was followed. A SYLGARD™ 184 Silicone elastomer was mixed with a curing agent in 10:1 ratio. Trapped bubbles during the mixing were degassed by vacuum oven. The clear transparent elastomer solution was gently poured over the diffraction lithography microneedle array and cured at 80° C. for an hour. After cooling down to the room temperature, the diffraction lithography microneedle array was detached from the solidified PDMS to yield the PDMS mold as a negative template for the microneedle array (array of trenches/holes corresponding to the microneedles). The PLA molding process involved covering the PDMS mold with PLA pellets (1-2 mm). The sample was heated at 180° C. in an oven to melt down the PLA pellets to fill up the PDMS mold trenches. After cooling down to a room temperature, the PLA microneedle array was detached from the PDMS mold to complete the PLA microneedle process.

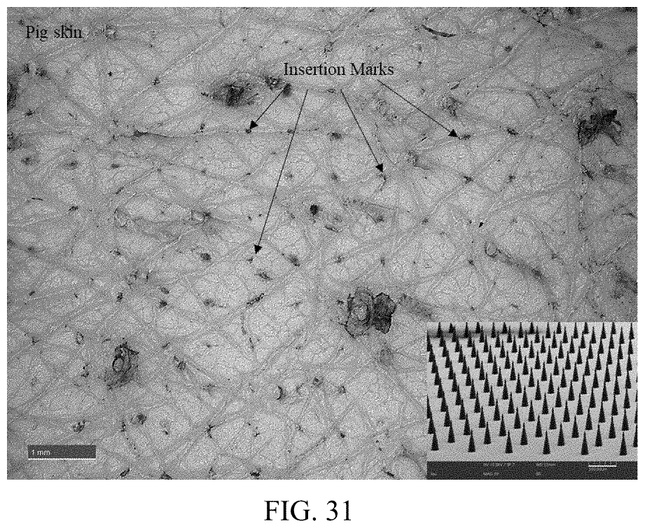

The PLA microneedles array was inserted into the pig skin by pressing at the back of the PLA substrate using thumb force. The insertion area was tinted with blue tissue marking dye (CDI's Tissue Marking Dye, Cancer Diagnostics, Inc.) for visualization. FIG. 31 shows the result of insertion test on the pig skin.

### VI. Force Displacement Test

To further understand the mechanical strength of the microneedles, force-displacement test was conducted. A 3×3 microneedle array was fabricated and used as a template for micromolding to create a PLA microneedle array using a two-step molding process including PDMS molding and PLA molding. The characteristics of the solid straight microneedles array are listed below:
Diameter=133 μm
Height=526 μm
Tip Diameter=40 μm
Tip Height=134 μm
Material =PLA
Number of needles=9 (3×3 array)

The 3×3 microneedles array was placed up straight while the force gauge was slowly moved downwards at a speed of 1.2 mm/min. The microneedle tip was first compressed by the force gauge and completely bent with a measured force of 0.552 N and 0.0613 N per needle.

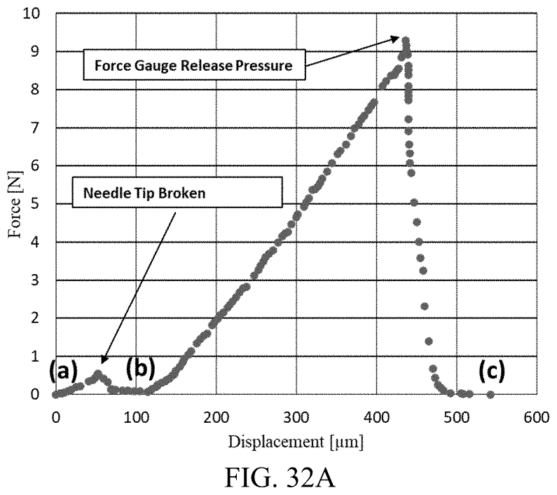

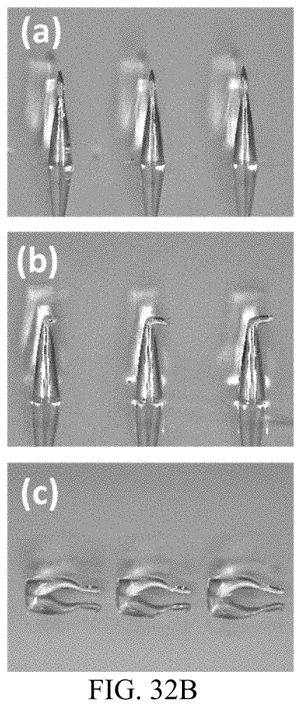

The force gauge continued to compress the body of the microneedles until it reached the maximum programmed time frame. The maximum compression force measured by the force gauge on the microneedle bodies was 9.284 N and 1.0316 N per needle, the total compressed displacement was measured to be 436 μm, which also matches with the compressed height measured using microscope afterwards. The data is shown in FIG. 32A, with corresponding images of the microneedles at each stage in FIG. 32B.

| Force Gauge Release Pressure | Needle Tip Broken |
|---|---|
| Displacement = 436 um | Displacement = 52.5 um |
| Force = 9.284N | Force = 0.552N |
| Force per Needle = 1.0316N | Force per Needle = 0.0613N |

A 2×2 solid inclined microneedle was fabricated using the diffraction lithography method. The fabrication process was very identical to the fabrication process for solid straight microneedles except an additional inclined angle was introduced during the UV exposure process. The inclined angle for the microneedles was measured to 14°. In this test, the microneedles were not converted into PLA. The conditions of the solid inclined microneedles are listed below:
Diameter=300 μm
Height=900 μm
Tip Diameter=90 μm
Tip Height=266 ρm
Material=Surgical Guide Resin Number of needles=4 (2×2 array)

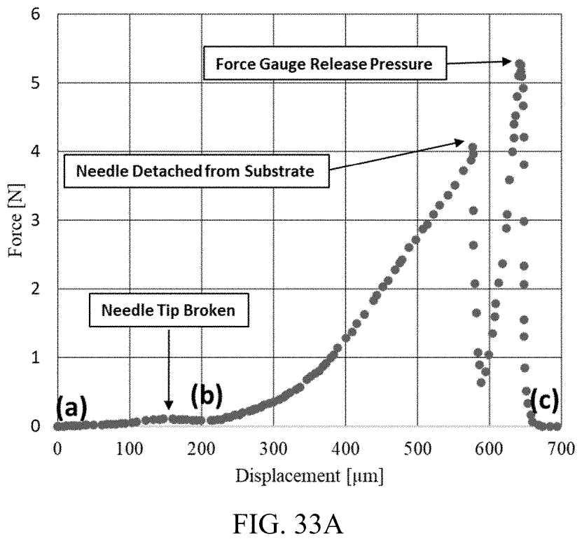

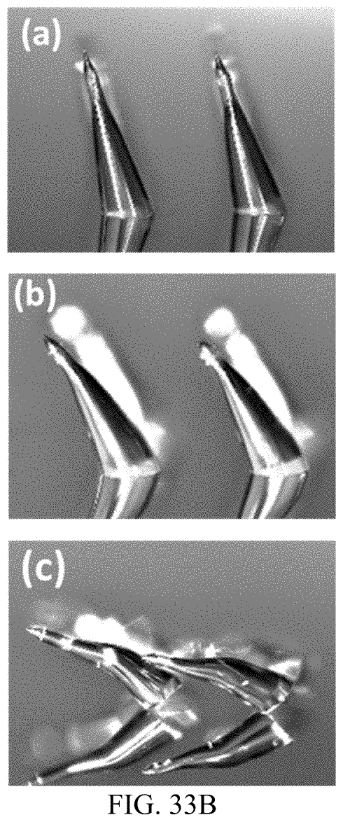

The 2×2 inclined microneedles array was placed up straight while the force gauge was slowly moved downwards at a speed of 1.2 mm/min. The microneedle tip was first compressed by the force gauge and completely bent with a measured force of 0.106 N and 0.0265 N per needle. The force gauge then continued to compress the body of the microneedles until the microneedles detached from the substrate. Since these microneedles were not converted into PLA, the adhesion force between the needles and the substrate are weaker than the solid straight needle test above. The measured detachment force from the substrate was 4.06 N and 1.015 N per needle. The force gauge continued the compression to the side way of the microneedle bodies until the maximum programmed time frame has reached, the measured force at this point was 5.276 N and 1.319 N per needle. The data is shown in FIG. 33A, with corresponding images of the microneedles at each stage in FIG. 33B.

| Force Gauge | Needle Detached from | |
|---|---|---|
| Release Pressure | Substrate | Needle Tip Broken |
| Displacement = 642 um | Displacement = 577 um | Displacement = 147 um |
| Force = 5.276N | Force = 4.06N | Force = 0.106N |
| Force per Needle = | Force per Needle = | Force per Needle = |
| 1.319N | 1.015N | 0.0265N |

Lastly, the detachment force required to detach inclined microneedles from two directions was investigated. Here, if the force gauge is moving against the inclined direction of the microneedle, it is called out-phase compression, whereas if the force gauge is moving towards the inclined direction of the microneedle, it is called in-phase compression. The conditions of the solid inclined microneedles are listed below:
Diameter=300 μm
Height=900 μm
Tip Diameter=90 μm
Tip Height=266 μm
Material=Surgical Guide Resin
Number of needles=1

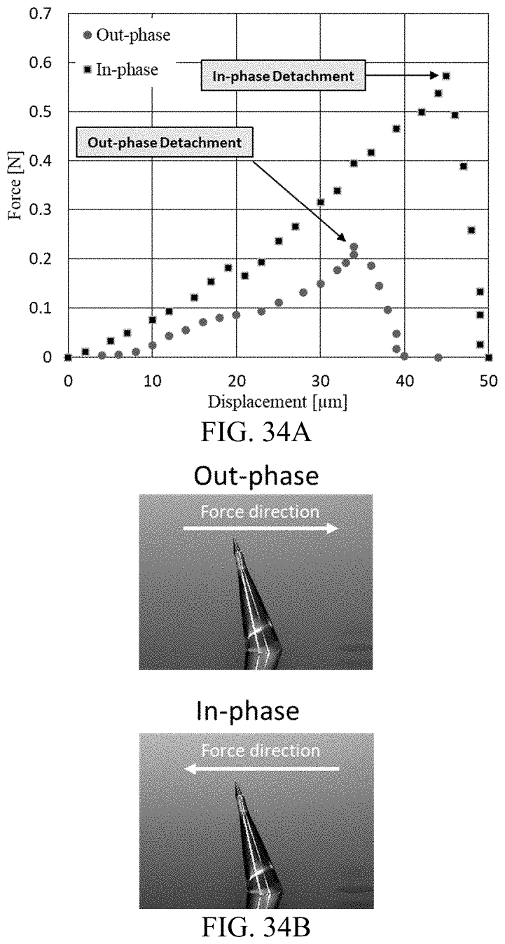

While the force gauge was still moving downwards with a speed of 1.2 mm/min, the microneedle was positioned as illustrated in FIG. 34B, depending on whether in-phase or out-phase was being tested. The out-phase detachment force was measured to be 0.224 N while the in-phase detachment force was measured to be 0.574 N. The data is shown in FIG. 34A, with corresponding images of the microneedles showing the direction of compression in FIG. 34B.

| Out-phase Detachment | In-phase Detachment |
|---|---|
| Displacement = 34 um | Displacement = 45 um |
| Force = 0.224N | Force = 0.574N |

What is claimed is:

1. A lithography method for fabricating a plurality of micro-sized structures with converging tips, the method comprising:
providing a substrate having an upper surface and a backside surface, wherein said substrate comprises a pattern having open areas configured to permit transmission of radiation and solid areas configured to prevent transmission of radiation;
forming a layer of liquid-state photosensitive resin on said upper surface;
exposing said liquid-state photosensitive resin to radiation through said substrate from the backside surface over a first period of time to yield light-exposed portions of said liquid-state photosensitive resin, wherein said light-exposed portions are crosslinked and/or polymerized into respective initial solid-state resin structures on said upper surface in alignment with said open areas, said initial solid-state resin structures having an increased refractive index as compared to said liquid-state photosensitive resin, such that each initial solid-state resin structure acts as a waveguide directing said radiation passing through said open areas of said pattern to a converging point thereby forming solid-state resin structures with tapered sidewalls and converging tips; and
contacting the coating layer with a solvent system so as to remove non-light exposed portions of said liquid-state photosensitive resin to leave behind a plurality of said micro-sized solid-state resin structures with tapered sidewalls and converging tips across said upper surface of said substrate.

2. The method of claim 1, wherein said open areas are apertures having a geometric shape selected from the group consisting of circular, rectangular, polygonal, and star.

3. The method of claim 2, wherein said apertures have a size of from about 1 μm to about 1,000 μm.

4. The method of claim 2, wherein said open areas have central portions that are opaque to prevent radiation from passing through the central portion of each aperture.

5. The method of claim 4, wherein said micro-sized structures with converging tips have a hollow shaft.

6. The method of claim 1, wherein said pattern is a photomask adjacent said upper surface and/or said backside surface of said substrate. The method of claim 1, wherein said pattern is integrally formed with said substrate.

8. The method of claim 6, wheren the pattern comprises an array of a plurality of spaced-apart apertures distributed across the substrate.

9. The method of claim 7, wherein the pattern comprises an array of a plurality of spaced-apart apertures distributed across the substrate.

10. The method of claim 1, wherein said layer of liquid-state photosensitive resin has a thickness that is taller than the height of said micro-sized solid-state resin structures.

11. The method of claim 1, wherein said layer of liquid-state photosensitive resin has a thickness ranging from about 50 μm to about 9 mm.

12. The method of claim 1, wherein said radiation is light at a wavelength of from about 300 nm to about 450 nm.

13. The method of claim 1, wherein said radiation is exposed through a collimating lens such that the direction of propagation of energy flow from the source of radiation is parallel and enters the substrate at an incident angle perpendicular to the backside surface of the substrate.

14. The method of claim 1, wherein said exposing step is carried out for a time period of from about 1 second to about 1 hour.

15. The method of claim 1, wherein said micro-structures are formed with a single exposing step, wherein said method does not include more than one exposing step.

16. The method of claim 1, wherein said exposing step comprises said first period of time and further comprises at least a second period of time continuous with said first period of time, wherein said micro-sized solid-state resin structures with tapered sidewalls and converging tips have a first height after said first period of time, and wherein said micro-sized solid-state resin structures with tapered sidewalls and converging tips have a second height after said second period of time that is greater than said first height.

17. The method of claim 16, wherein exposure to radiation during said second period of time induces further crosslinking and/or photopolymerizing in regions of the resin layer adjacent to the converging tips of said initial micro-sized solid-state resin structures of the first height, thereby forming one or more additional harmonic structures on said initial micro-sized solid-state resin structures.

18. The method of claim 17, wherein said one or more additional harmonic structures have sidewalls with alternating inclining and declining angles ultimately converging at respective tips.

19. The method of claim 1, wherein micro-sized solid-state resin structures comprise respective shafts having cross-sectional geometries selected from the group consisting of circular, rectangular, polygonal, and oblong, and wherein a combination of any of the foregoing geometries may be provided in a single micro-structure array across said substrate.

20. The method of claim 1, wherein micro-sized solid-state resin structures each have a base size ranging from about 5 μm to about 1,000 μm, and a height ranging from about 30 μm to about 9 mm.

21. The method of claim 1, wherein said substrate is substantially planar, and wherein said substrate remains stationary during said exposing.

22. The method of claim 1, further comprising applying one or more intervening layers to said substrate before applying said photosensitive resin layer.

23. The method of claim 1, further comprising using said plurality of said micro-sized solid-state resin structures as a template for micromolding.

24. A method for delivering of active agent across a biological barrier, the method comprising the steps of:
puncturing the biological barrier with a plurality of microneedles formed according to the method of claim 1.

25. The method of claim 24, wherein said biological barrier is selected from the group consisting of stratum corneum, epidermis, dermis, and combinations thereof.

26. A lithography method for fabricating a plurality of micro-sized structures with two or more harmonic structures using a single exposing step, said method comprising:
providing a substrate having an upper surface and a backside surface, wherein said substrate comprises a pattern having open areas configured to permit transmission of radiation and solid areas configured to prevent transmission of radiation;
forming a layer of liquid-state photosensitive resin on said upper surface;
exposing said liquid-state photosensitive resin to radiation through said substrate from the backside surface over a first period of time, wherein initial light-exposed portions of said liquid-state photosensitive resin are crosslinked and/or polymerized into initial solid-state resin structures, said initial solid-state resin structures self-focusing said radiation into a converging beam path, such that continued exposing over a second period of time yield secondary light-exposed portions adjacent said initial light-exposed portions, said secondary light exposed portions being crosslinked and/or polymerized into secondary and, optionally, tertiary harmonic structures with converging tips adjacent said initial solid-state resin structures; and
contacting the layer with a solvent system so as to remove non-light exposed portions of said liquid-state photosensitive resin to yield a plurality of micro-sized solid-state resin structures with two or more harmonic structures across said upper surface of said substrate.
